(19) United States
(12) Patent Application Publication
(10) Pub. No.: US 2026/0124446 A1
(43) Pub. Date: May 7, 2026

(54) LOW-FREQUENCY TREATMENT INSTRUMENT AND LOW-FREQUENCY TREATMENT INSTRUMENT UNIT

(71) Applicant: OMRON HEALTHCARE Co., Ltd., Kyoto (JP)

(72) Inventors: Yu KAWASHIMA, Kyoto (JP); Tomoyuki NISHIDA, Kyoto (JP)

(21) Appl. No.: 19/427,372

(22) Filed: Dec. 19, 2025

Related U.S. Application Data
(63) Continuation of application No. PCT/JP2024/035258, filed on Oct. 2, 2024.

(30) Foreign Application Priority Data
Dec. 12, 2023 (JP) 2023-209020

(51) Int. Cl. A61N 1/36; A61N 1/04

(52) U.S. Cl. CPC A61N 1/3603; A61N 1/0492

(57) ABSTRACT

A low-frequency treatment device includes an electrode portion configured to supply a current to a body surface, and a light irradiation unit configured to emit light for confirming an attachment position at which the electrode portion is attached to the body surface. The light irradiation unit is disposed to be able to emit light parallel to the electrode portion in a facing state in which the electrode portion faces the body surface at a distance from the body surface.

### CROSS-REFERENCE TO RELATED APPLICATIONS

This application is the U.S. national stage application filed pursuant to 35 U.S.C. 365(c) and 120 as a continuation of International Patent Application No. PCT/JP2024/035258, filed October 2, 2024, which application claims priority to Japanese Patent Application No. 2023-209020, filed December 12, 2023, which applications are incorporated herein by reference in their entireties.

### FIELD

The present disclosure relates to a low-frequency treatment device and a low-frequency treatment device unit.

### BACKGROUND ART

As a conventional low-frequency treatment device, JP 04-197271 A (Patent Document 1) discloses a configuration in which a plurality of light-emitting diodes for indicating a selected treatment mode are provided on an upper surface side of a housing in a low-frequency treatment device.

### CITATION LIST

### Patent Literature

Patent Document 1: JP 04-197271 A

### SUMMARY

### Technical Problem

The low-frequency treatment device provides stimulation by outputting an electrical signal to a muscle via an electrode portion, the electrode portion being placed in contact with a surface of the body such as the abdomen or the back.

The surface of the body can be divided into a plurality of cutaneous sensation areas (dermatomes) associated with spinal nerves, and the therapeutic effect can be improved by applying electricity to a predetermined dermatome.

However, when the electrode portion is attached to the back or the like, since it is difficult to visually confirm the back side, the height position, the position in a circumferential direction, and the like of the electrode portion are not easily grasped. Therefore, it is difficult to attach the electrode portion to an appropriate position.

The present disclosure is made in view of the above problems, and an object of the present disclosure is to provide a low-frequency treatment device and a low-frequency treatment device unit that enable an electrode portion to be guided to an appropriate position on a living body surface.

### Solution To Problem

A low-frequency treatment device based on the present disclosure includes an electrode portion configured to supply a current to a body surface, and a light irradiation unit configured to emit light for confirming an attachment position at which the electrode portion is attached to the body surface. The light irradiation unit is disposed to be able to emit light parallel to the electrode portion in a facing state in which the electrode portion faces the body surface at a distance from the body surface.

Note that the term "parallel" does not only mean "completely parallel", but also means "substantially parallel", which allows for a slight deviation due to a manufacturing error such as a tolerance.

According to the above configuration, before the electrode portion is attached to the body surface, the light emitted from the light irradiation unit in the direction parallel to the electrode portion is emitted to a predetermined irradiated object in a state in which the electrode portion faces the body surface. Accordingly, the position of the light emitted to the irradiated object is confirmed, and thus, the position of the electrode portion can be confirmed. Therefore, the electrode portion can be attached to an appropriate position on the body surface.

In the low-frequency treatment device based on the present disclosure, the light irradiation unit may be located on a side opposite to a side on which the body surface of a user is located with respect to the electrode portion in the facing state.

According to the configuration described above, when attaching the electrode portion to the body surface, the light irradiation unit can be prevented from entering between the electrode portion and the body surface.

The low-frequency treatment device based on the present disclosure may include a main body portion from which a current is supplied to the electrode portion. The light irradiation unit may be disposed on the main body portion.

According to the above configuration, the light irradiation unit can be installed at a stable position, and the position of the light emitted from the light irradiation unit can also be stabilized.

In the low-frequency treatment device based on the present disclosure, the main body portion may include a bottom surface portion that faces the body surface in the facing state, and a first sidewall portion and a second sidewall portion that are disposed to stand from an outer edge of the bottom surface portion and face each other with a distance therebetween. The light irradiation unit may include a first light irradiation unit disposed on the first sidewall portion and a second light irradiation unit disposed on the second sidewall portion. In this case, the first light irradiation unit and the second light irradiation unit may be arranged such that positions thereof in an orthogonal direction orthogonal to a direction in which the first sidewall portion and the second sidewall portion face each other are coincident with each other.

According to the above configuration, by confirming a position in which the light emitted from the first light irradiation unit installed on the first sidewall portion is emitted to the irradiated object and a position in which the light emitted from the second light irradiation unit installed on the second sidewall portion is emitted to another irradiated object, the position of the electrode portion can be adjusted while grasping the position (more specifically, the degree of rotation) of the electrode portion. For example, when the direction in which the first sidewall portion and the second sidewall portion face each other is inclined from the horizontal direction, the direction in which the first sidewall portion and the second sidewall portion face each other can be set to the horizontal direction by adjusting the irradiation position such that the irradiation position from the first light irradiation unit and the irradiation position from the second light irradiation unit are at the same height position in the vertical direction. The electrode portion can be disposed at an appropriate position.

In the low-frequency treatment device based on the present disclosure, the main body portion may include a bottom surface portion that faces the body surface in the facing state and a peripheral wall portion disposed along an outer edge of the bottom surface portion. The light irradiation unit may include a first light irradiation unit disposed at a first position of the peripheral wall portion, and a second light irradiation unit disposed at a second position rotated by 90 degrees from the first position in a circumferential direction of the peripheral wall portion.

According to the above configuration, by confirming two irradiation positions of lights emitted in directions different by 90 degrees, the position of the electrode portion can be adjusted while grasping the position (more specifically, the degree of rotation) of the electrode portion.

The low-frequency treatment device based on the present disclosure may include a gripping portion disposed on the main body portion. The gripping portion may be arranged such that the light emitted from the light irradiation unit to a hand of the user gripping the gripping portion does not hit the user in a state in which the user grips the gripping portion.

According to the above configuration, the attachment position of the electrode portion can be adjusted while gripping the gripping portion.

In the low-frequency treatment device based on the present disclosure, the light irradiation unit may be arranged such that the light emitted from the light irradiation unit overlaps at least a part of the electrode portion when viewed from a normal direction of the electrode portion.

According to the above configuration, since the light is emitted from the light irradiation unit so as to overlap at least a part of the electrode portion when viewed from the normal direction of the electrode portion, the position of the electrode portion can be confirmed by confirming the position irradiated with the light.

The low-frequency treatment device based on the present disclosure may include a control unit configured to operate an operation of the light irradiation unit. The control unit may control the light irradiation unit from an on state to an off state when the current is supplied to the electrode portion.

According to the above configuration, the light irradiation unit does not emit light in accordance with the start of treatment of the low-frequency treatment device, and thus power consumption can be suppressed.

A low-frequency treatment device unit based on the present disclosure includes the low-frequency treatment device described above, and an auxiliary member on which an indicator of the attachment position is indicated. Light is emitted from the light irradiation unit toward the auxiliary member, thereby enabling the attachment position to be confirmed.

According to the above configuration, the indicator of the attachment position can be confirmed by the position of the light, and the electrode portion can be disposed at a more appropriate position.

### Advantageous Effects Of Invention

According to the present disclosure, a low-frequency treatment device and a low-frequency treatment device unit that enable an electrode portion to be guided to an appropriate position on a living body surface can be provided.

### BRIEF DESCRIPTION OF DRAWINGS

Various embodiments are disclosed, by way of example only, with reference to the accompanying schematic drawings in which corresponding reference symbols indicate corresponding parts.

FIG. 1 is a perspective view illustrating a low-frequency treatment device according to a first embodiment.

FIG. 2 is a schematic front view of the low-frequency treatment device according to the first embodiment.

FIG. 3 is a diagram of a state of attaching the low-frequency treatment device according to the first embodiment to the back side as viewed from above.

FIG. 4 is a diagram of a state of attaching the low-frequency treatment device according to the first embodiment to the back side as viewed from the rear side.

FIG. 5 is a graph illustrating an on/off state of current supply to a light irradiation unit and electrode portions of the low-frequency treatment device according to the first embodiment.

FIG. 6 is a schematic plan view of a low-frequency treatment device according to a second embodiment.

FIG. 7 is a schematic plan view of a low-frequency treatment device according to a third embodiment.

FIG. 8 is a schematic plan view of a low-frequency treatment device according to a fourth embodiment.

FIG. 9 is a schematic plan view a low-frequency treatment device according to a fifth embodiment.

FIG. 10 is a schematic diagram illustrating a low-frequency treatment device unit according to a sixth embodiment.

### DESCRIPTION OF EMBODIMENTS

Embodiments of the present disclosure will be described in detail below with reference to the drawings. In the embodiments described below, identical or common components are denoted by the same reference numerals in the drawings, and repeated descriptions thereof are omitted.

### First Embodiment

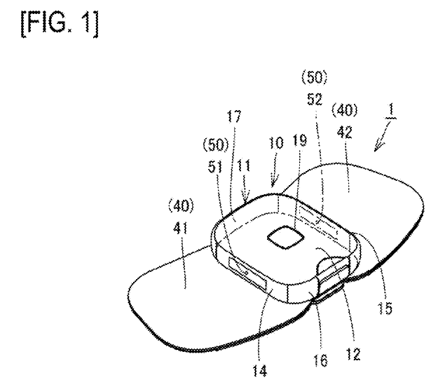

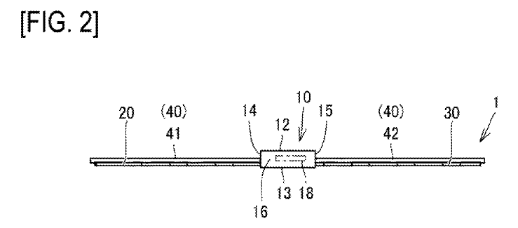

FIG. 1 is a perspective view illustrating a low-frequency treatment device according to a first embodiment. FIG. 2 is a schematic front view of the low-frequency treatment device according to the first embodiment. A low-frequency treatment device 1 according to the first embodiment will be described with reference to FIG. 1 and FIG. 2.

The low-frequency treatment device 1 includes a main body portion 10, electrode portions 20 and 30, a holding portion 40, and a light irradiation unit 50. The low-frequency treatment device 1 supplies a current to the body surface and relieves pain of a user, for example, by supplying a low-frequency pulse current to the electrode portions 20 and 30 placed in contact with the body surface of the user. The frequency of low-frequency pulse current is, for example, 1 Hz or higher and 1200 Hz or lower.

The main body portion 10 includes a housing case 11 and an operation unit 19. A control unit 18 is housed inside the housing case 11. The control unit 18 is electrically connected to the electrode portions 20 and 30 via an electrical path (not illustrated), and controls a current, a voltage, and the like flowing through the electrode portions 20 and 30. The control unit 18 also controls the operation of the light irradiation unit 50. The control unit 18 is formed of a circuit board or the like.

The operation unit 19 is disposed on the housing case 11. The operation unit 19 includes, for example, a measurement switch and the like. The operation unit 19 is not limited to a push type switch and may be, for example, a pressure sensitive type (resistance type) or a proximity type (electrostatic capacitive type) touch panel type switch or the like. The housing case 11 may be provided with a display unit separately from the operation unit 19.

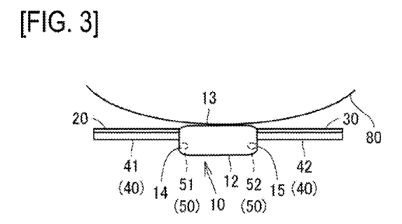

The housing case 11 has a box shape. The housing case 11 includes a top surface portion 12, a bottom surface portion 13, a first sidewall portion 14, a second sidewall portion 15, a first wall portion 16, and a second wall portion 17. The top surface portion 12 and the bottom surface portion 13 face each other in a predetermined first direction. At least a part of the bottom surface portion 13 comes into contact with a body surface 80 (see FIG. 3) when the low-frequency treatment device 1 is used. The first sidewall portion 14, the second sidewall portion 15, the first wall portion 16, and the second wall portion 17 constitute a peripheral wall portion of the housing case 11. The first sidewall portion 14 and the second sidewall portion 15 are disposed so as to stand from an outer edge of the bottom surface portion 13 and face each other with a distance therebetween. The first sidewall portion 14 and the second sidewall portion 15 face each other in a second direction orthogonal to the first direction. The first wall portion 16 and the second wall portion 17 are disposed so as to stand from the outer edge of the bottom surface portion 13 and face each other with a distance therebetween. The first wall portion 16 and the second wall portion 17 face each other in a third direction orthogonal to the first direction and the second direction.

The electrode portions 20 and 30 are spaced apart from each other in the second direction and disposed such that the main body portion 10 is located between the electrode portion 20 and the electrode portion 30 in a front view. The electrode portion 20 is disposed on one side in the second direction with respect to the main body portion 10. The electrode portion 30 is disposed on the other side in the second direction with respect to the main body portion 10.

The electrode portions 20 and 30 are portions for applying a current to the user. The electrode portions 20 and 30 are disposed to be attachable with adhesive force to the body surface of the user. The electrode portions 20 and 30 include adhesive portions. The electrode portions 20 and 30 may be, for example, gel pads or may be laminate bodies each formed of an adhesive layer and a conductive member. By supplying a current to the electrode portions 20 and 30 in a state in which the electrode portions 20 and 30 are in contact with the body surface of the user, the current can be applied to the user.

The holding portion 40 holds the electrode portions 20 and 30. The holding portion 40 has, for example, flexibility and is disposed to be deformable along the body surface. For example, a cloth or the like having insulating properties can be employed as the holding portion 40.

The holding portion 40 is fixed to, for example, the main body portion 10. The holding portion 40 includes a first holding portion 41 and a second holding portion 42. The first holding portion 41 holds the electrode portion 20. The shape of the first holding portion 41 is substantially identical to the shape of the electrode portion 20. The second holding portion 42 holds the electrode portion 30. The shape of the second holding portion 42 is substantially identical to the shape of the electrode portion 30. The first holding portion 41 and the second holding portion 42 may be connected by a connection portion.

The light irradiation unit 50 is disposed so as to be able to emit light. The light irradiation unit 50 includes a light source and is configured to emit linear light. For example, a laser beam is emitted from the light irradiation unit 50. A slit having an opening may be disposed at a front part of the light source, and the laser beam may be emitted in a shape corresponding to the shape of the opening of the slit. The opening shape may be a rectangular shape, a polygonal shape, or a cross shape. The opening shape may be an oval shape such as a circular shape, an elliptical shape, or a track shape.

The light irradiation unit 50 is disposed on the main body portion 10. Accordingly, the light irradiation unit 50 can be installed at a stable position, and the light emitted from the light irradiation unit 50 can also be stabilized. The light irradiation unit 50 includes a first light irradiation unit 51 and a second light irradiation unit 52.

The first light irradiation unit 51 is disposed on the first sidewall portion 14. The second light irradiation unit 52 is disposed on the second sidewall portion 15. The irradiation direction of the first light irradiation unit 51 and the irradiation direction of the second light irradiation unit 52 are different from each other. In the present embodiment, the second light irradiation unit 52 emits light in an opposite direction to the irradiation direction of the first light irradiation unit 51.

FIG. 3 is a diagram of a state of attaching the low-frequency treatment device according to the first embodiment to the back side as viewed from above.

As illustrated in FIG. 3, when attaching the low-frequency treatment device 1 to the back side, first, the main body portion 10 is brought into contact with the body surface 80 on the back side. Specifically, the bottom surface portion 13 of the main body portion 10 is brought into contact with the body surface 80. In this state, the electrode portions 20 and 30 are separated from the body surface 80. In other words, the electrode portions 20 and 30 face the body surface 80 at a distance therefrom. In the facing state in which the electrode portions 20 and 30 face the body surface 80 as just described, the light irradiation unit 50 is located on a side opposite to a side on which the body surface 80 is located with respect to the electrode portions 20 and 30. Accordingly, when attaching the electrode portions 20 and 30 to the body surface 80, the light irradiation unit 50 can be prevented from entering between the electrode portions 20 and 30 and the body surface 80. In other words, the light can be reliably applied from the light irradiation unit 50 toward the outside.

In the facing state, the first light irradiation unit 51 and the second light irradiation unit 52 are disposed such that the positions thereof in an orthogonal direction (the third direction) orthogonal to the direction (the second direction) in which the first sidewall portion 14 and the second sidewall portion 15 face each other are coincident with each other. Note that being "coincident with each other" allows for manufacturing errors such as tolerances, and means that the positions are substantially the same.

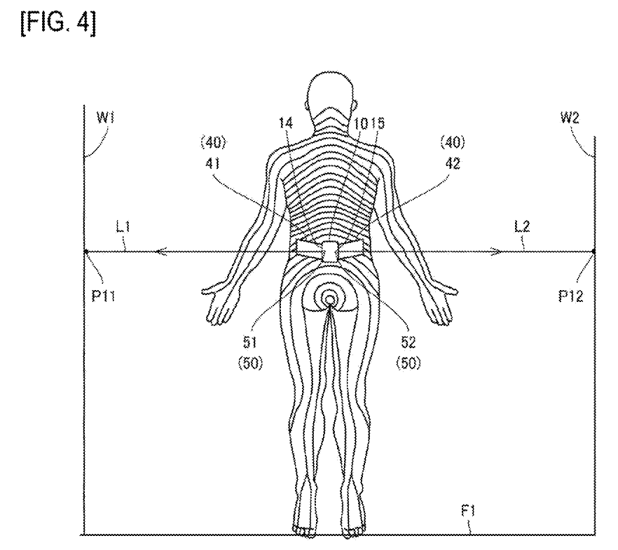

FIG. 4 is a diagram of a state of attaching the low-frequency treatment device according to the first embodiment to the back side as viewed from the rear side. In FIG. 4, in order to clearly illustrate the dermatome, a state in which the user does not grip the low-frequency treatment device 1 is illustrated for convenience. When actually attaching the low-frequency treatment device 1 to the back side, the user positions the low-frequency treatment device 1 while gripping the low-frequency treatment device 1 with the hand. The low-frequency treatment device 1 (more specifically, the electrode portions 20 and 30) is arranged on a dermatome suitable for treatment of a plurality of cutaneous sensation areas (dermatomes), and thus treatment can be effectively performed. Therefore, positioning of the low-frequency treatment device 1 is important.

As illustrated in FIG. 4, when positioning the low-frequency treatment device 1, light is emitted from the light irradiation unit 50, and the position of the light emitted to an irradiated object is confirmed. Here, wall portions W1 and W2 in a room will be described as an example of the irradiated object, but the irradiated object is not limited to the wall portions in the room.

The light irradiation unit 50 is disposed so as to be able to emit light parallel to the electrode portions 20 and 30 in the facing state in which the electrode portions 20 and 30 face the body surface 80 at a distance therefrom. When viewed from the normal direction of the electrode portions 20 and 30, the light irradiation unit 50 is disposed such that the light emitted from the light irradiation unit 50 overlaps at least a part of the electrode portions 20 and 30. Accordingly, the positions (more specifically, the height positions) of the electrode portions 20 and 30 can be easily confirmed by confirming the position irradiated with the light.

The first light irradiation unit 51 emits a light L1 toward one side in the second direction (a side opposite to a side on which the second sidewall portion 15 is located), and the wall portion W1 is illuminated with the light. The second light irradiation unit 52 emits a light L2 toward the other side in the second direction (a side opposite to a side on which the first sidewall portion 14 is located), and the wall portion W2 is illuminated with the light.

The user can grasp the positions (specifically, the height positions in a vertical direction and the degree of rotation) of the electrode portions 20 and 30 by confirming an irradiation position P11 in which the wall portion W1 is irradiated with the light L1 emitted from the first light irradiation unit 51 and an irradiation position P12 in which the wall portion W2 is irradiated with the light L2 emitted from the second light irradiation unit 52.

For example, when the second direction is inclined from a horizontal direction, the irradiation positions P11 and P12 are adjusted such that the irradiation position P11 and the irradiation position P12 are at the same height position in the vertical direction, and thus the second direction can set to be the horizontal direction. In addition, the positions of the irradiation positions P11 and P12 in the vertical direction (the height positions from a floor surface F1) are adjusted, and thus the height positions of the electrode portions 20 and 30 on the back side can be adjusted. As just described, the positions of the electrode portions 20 and 30 are grasped, and the electrode portions 20 and 30 are brought into contact with the body surface 80. Thus, the electrode portions 20 and 30 can be attached to appropriate positions.

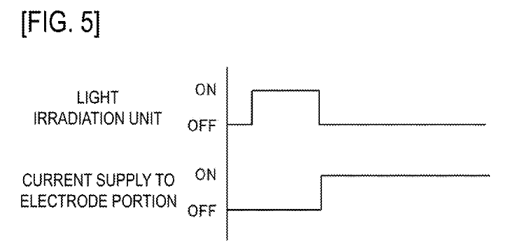

FIG. 5 is a graph illustrating an on/off state of current supply to the light irradiation unit and the electrode portions of the low-frequency treatment device according to the first embodiment.

As illustrated in FIG. 5, the control unit 18 controls the light irradiation unit 50 from the on state to the off state when a current is supplied to the electrode portions 20 and 30. Accordingly, the light irradiation unit does not emit light in accordance with the start of treatment of the low-frequency treatment device, and thus power consumption can be suppressed. In addition, the light is emitted from the light irradiation unit 50 at the time of position adjustment, and thus the position adjustment can be easily performed.

### Second Embodiment

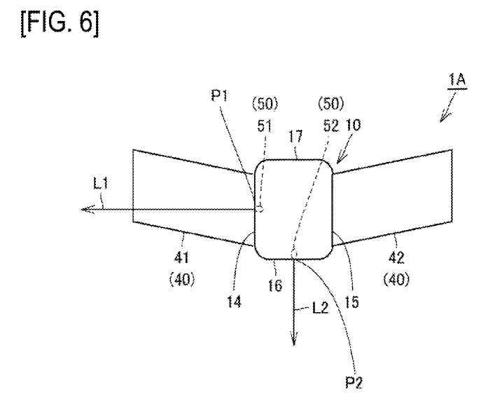

FIG. 6 is a schematic plan view of a low-frequency treatment device according to a second embodiment. A low-frequency treatment device 1A according to the second embodiment will be described with reference to FIG. 6.

As illustrated in FIG. 6, the low-frequency treatment device 1A according to the second embodiment is different from the low-frequency treatment device 1 according to the first embodiment in the position of the second light irradiation unit 52. Other configurations are almost the same.

The first light irradiation unit 51 is disposed at a first position P1 of the peripheral wall portion of the main body portion 10, and the second light irradiation unit 52 is disposed at a second position P2 rotated by 90 degrees from the first position P1 in a circumferential direction of the peripheral wall portion. Specifically, for example, the first light irradiation unit 51 is disposed on the first sidewall portion 14, and the second light irradiation unit 52 is disposed on the first wall portion 16.

Even with such a configuration, the low-frequency treatment device 1A according to the second embodiment can achieve effects similar to those of the low-frequency treatment device 1 according to the first embodiment. By confirming two irradiation positions of lights emitted in directions different by 90 degrees, the positions of the electrode portions 20 and 30 can be adjusted while grasping the positions (more specifically, the degree of rotation) of the electrode portions 20 and 30.

### Third Embodiment

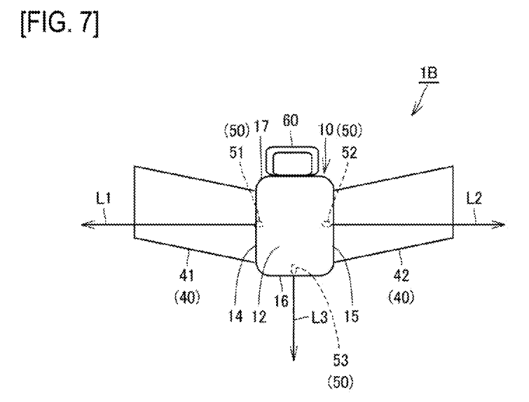

FIG. 7 is a schematic plan view of a low-frequency treatment device according to a third embodiment. A low-frequency treatment device 1B according to the third embodiment will be described with reference to FIG. 7.

As illustrated in FIG. 7, the low-frequency treatment device 1B according to the third embodiment is different from the low-frequency treatment device 1 according to the first embodiment in that a gripping portion 60 is provided and that the light irradiation unit 50 includes a third light irradiation unit 53. Other configurations are almost the same.

The first light irradiation unit 51 and the second light irradiation unit 52 are disposed at a pitch of 180 degrees in the circumferential direction of the peripheral wall portion, and the third light irradiation unit 53 is disposed at the center between the first light irradiation unit 51 and the second light irradiation unit 52 in the circumferential direction. The third light irradiation unit 53 is disposed at a position rotated by 90 degrees in the circumferential direction from each of the first light irradiation unit 51 and the second light irradiation unit 52.

Specifically, the first light irradiation unit 51 is disposed on the first sidewall portion 14, and the second light irradiation unit 52 is disposed on the second sidewall portion 15. The third light irradiation unit 53 is disposed on the first wall portion 16. The third light irradiation unit 53 is disposed so as to be able to emit a light L3 to one side in the third direction (a side opposite to a side on which the second wall portion 17 is located).

In the above description, a case where three light irradiation units are provided is illustrated, but the number of light irradiation units may be two light irradiation units.

The gripping portion 60 is disposed on the main body portion 10. The gripping portion 60 is, for example, a handle and has a U-shape. The gripping portion 60 is arranged such that the light emitted from the light irradiation unit 50 to the hand of the user gripping the gripping portion 60 does not hit the user in a state in which the user grips the gripping portion 60. The gripping portion 60 is disposed on the second wall portion 17.

Even with such a configuration, the low-frequency treatment device 1B according to the third embodiment can achieve effects similar to those of the low-frequency treatment device 1 according to the first embodiment. Since the attachment positions of the electrode portions 20 and 30 can be adjusted while gripping the gripping portion 60, convenience can be improved. In addition, the positions of the electrode portions 20 and 30 are adjusted by using the lights L1, L2, and L3 from the first light irradiation unit 51, the second light irradiation unit 52, and the third light irradiation unit 53, and thus the position adjustment can be more appropriately performed.

### Fourth Embodiment

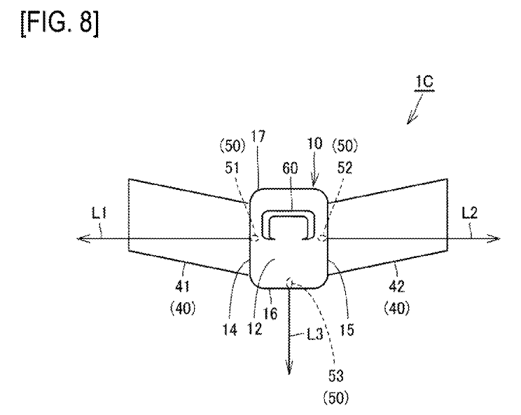

FIG. 8 is a schematic plan view of a low-frequency treatment device according to a fourth embodiment. A low-frequency treatment device 1C according to the fourth embodiment will be described with reference to FIG. 8.

As illustrated in FIG. 8, the low-frequency treatment device 1C according to the fourth embodiment is different from the low-frequency treatment device 1B according to the third embodiment in the position of the gripping portion 60. Other configurations are almost the same. The gripping portion 60 is disposed on the top surface portion 12 of the main body portion 10.

Even with such a configuration, the low-frequency treatment device 1C according to the fourth embodiment can achieve effects similar to those of the low-frequency treatment device 1B according to the third embodiment.

### Fifth Embodiment

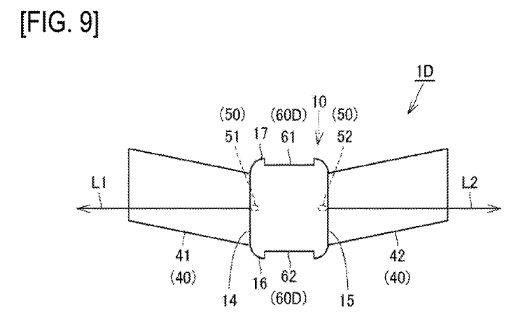

FIG. 9 is a schematic plan view a low-frequency treatment device according to a fifth embodiment. A low-frequency treatment device 1D according to the fifth embodiment will be described with reference to FIG. 9.

As illustrated in FIG. 9, the low-frequency treatment device 1D according to the fifth embodiment is different from the low-frequency treatment device 1B according to the third embodiment in that the third light irradiation unit 53 is not provided and a gripping portion 60D has a different configuration.

The gripping portion 60D is formed of recessed portions 61 and 62. The recessed portion 61 is disposed in the second wall portion 17. The recessed portion 61 is disposed so as to be recessed toward the side on which the first wall portion 16 is

located. The recessed portion 62 is disposed in the first wall portion 16. The recessed portion 62 is disposed so as to be recessed toward the side on which the second wall portion 17 is located. For example, when the user grips the main body portion 10, the user presses the recessed portion 61 with the thumb and supports the recessed portion 62 with the index finger, the middle finger, the ring finger, and the little finger.

Even with such a configuration, the low-frequency treatment device 1D according to the fifth embodiment can achieve effects similar to those of the low-frequency treatment device 1B according to the third embodiment.

### Sixth Embodiment

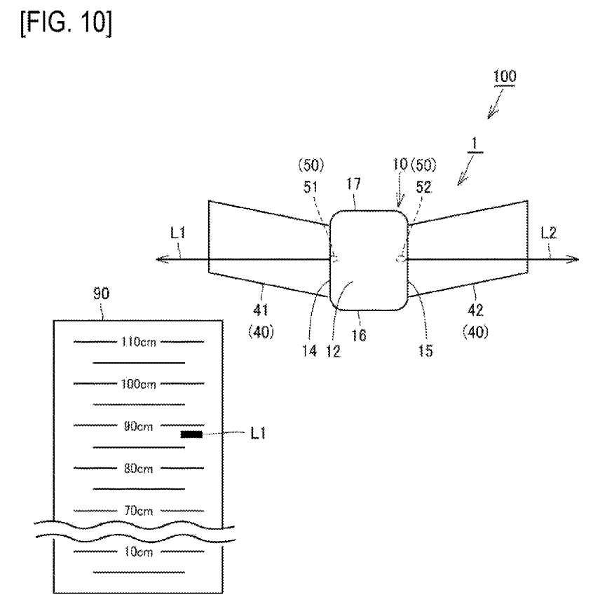

FIG. 10 is a schematic diagram illustrating a low-frequency treatment device unit according to a sixth embodiment. A low-frequency treatment device unit 100 according to the sixth embodiment will be described with reference to FIG. 10.

As illustrated in FIG. 10, the low-frequency treatment device unit 100 according to the sixth embodiment includes the low-frequency treatment device 1 and an auxiliary member 90. The low-frequency treatment device 1 is the same as that of the first embodiment, but any of the low-frequency treatment devices of the first to fifth embodiments described above can be adopted.

The auxiliary member 90 has an indicator of the attachment position. The auxiliary member 90 is, for example, a poster on which a height position from the floor surface F1 is indicated. The auxiliary member 90 may be a single or a plurality of members. By attaching the auxiliary member 90 to the above-described wall portions W1 and W2 and confirming the position of the light (L1 in FIG. 10) emitted to the auxiliary member 90, the position of the low-frequency treatment device 1 can be easily confirmed.

Although a case where the auxiliary member 90 is disposed separately from the low-frequency treatment device 1 is described above as an example, the auxiliary member 90 may be incorporated in the low-frequency treatment device 1. Specifically, a light transmitting portion on which a scale is marked may be disposed at the front part of the light source of the light irradiation unit 50, and an indicator such as a scale may be disposed so as to be projected on an irradiated object (for example, the wall portion W1) by the light emitted from the light source. In this case, by confirming the position of the light emitted to the projection image, the position of the low-frequency treatment device 1 can be easily confirmed. In addition, the auxiliary member 90 does not need to be disposed as a separate member, and thus costs can be reduced.

The embodiments disclosed herein are illustrative in all respects and are not intended as limitations. The scope of the present invention is defined by the claims, and all modified examples within the meaning and scope equivalent to the claims are intended to be included therein.

### REFERENCE SIGNS LIST

1, 1A, 1B, 1C, 1D Low-frequency treatment device, 10 Main body portion, 11 Housing case, 12 Top surface portion, 13 Bottom surface portion, 14 First sidewall portion, 15 Second sidewall portion, 16 First wall portion, 17 Second wall portion, 18 Control unit, 19 Operation unit, 20, 30 Electrode portion, 40 Holding portion, 41 First holding portion, 42 Second holding portion, 50 Light irradiation unit, 51 First light irradiation unit, 52 Second light irradiation unit, 53 Third light irradiation unit, 60, 60D Gripping portion, 61, 62 Recessed portion, 80 Body surface, 90 Auxiliary member, 100 Low-frequency treatment device unit, F1 Floor surface, L1, L2, L3 Light, P1 First position, P2 Second position, P11, P12 Irradiation position, W1, W2 Wall portion

What is claimed is:

1. A low-frequency treatment device, comprising:
an electrode portion configured to supply a current to a body surface; and
a light irradiation unit configured to emit light for confirming an attachment position at which the electrode portion is attached to the body surface,
wherein the light irradiation unit is disposed to be able to emit light parallel to the electrode portion in a facing state in which the electrode portion faces the body surface at a distance from the body surface.

2. The low-frequency treatment device according to claim 1, wherein the light irradiation unit is located on a side opposite to a side on which the body surface of a user is located with respect to the electrode portion in the facing state.

3. The low-frequency treatment device according to claim 2, comprising a main body portion from which a current is supplied to the electrode portion,
wherein the light irradiation unit is disposed on the main body portion.

4. The low-frequency treatment device according to claim 3, wherein
the main body portion includes a bottom surface portion that faces the body surface in the facing state, and a first sidewall portion and a second sidewall portion that are disposed to stand from an outer edge of the bottom surface portion and face each other with a distance therebetween,
the light irradiation unit includes a first light irradiation unit disposed on the first sidewall portion and a second light irradiation unit disposed on the second sidewall portion, and
the first light irradiation unit and the second light irradiation unit are arranged such that positions thereof in an orthogonal direction orthogonal to a direction in which the first sidewall portion and the second sidewall portion face each other are coincident with each other.

5. The low-frequency treatment device according to claim 3, wherein
the main body portion includes a bottom surface portion that faces the body surface in the facing state and a peripheral wall portion disposed along an outer edge of the bottom surface portion, and
the light irradiation unit includes a first light irradiation unit disposed at a first position of the peripheral wall portion, and a second light irradiation unit disposed at a second position rotated by 90 degrees from the first position in a circumferential direction of the peripheral wall portion.

6. The low-frequency treatment device according to claim 3, comprising a gripping portion disposed on the main body portion,
wherein the gripping portion is arranged such that the light emitted from the light irradiation unit to a hand of the user gripping the gripping portion does not hit the user in a state in which the user grips the gripping portion.

7. The low-frequency treatment device according to claim 1, wherein the light irradiation unit is arranged such that the light emitted from the light irradiation unit overlaps at least a part of the electrode portion when viewed from a normal direction of the electrode portion.

8. The low-frequency treatment device according to claim 1, comprising a control unit configured to operate an operation of the light irradiation unit,
wherein the control unit controls the light irradiation unit from an on state to an off state when the current is supplied to the electrode portion.

9. A low-frequency treatment device unit, comprising:
the low-frequency treatment device according to claim 1; and
an auxiliary member on which an indicator of the attachment position is indicated,
wherein light is emitted from the light irradiation unit toward the auxiliary member, thereby enabling the attachment position to be confirmed.
